(19) United States
(12) Patent Application Publication
(10) Pub. No.: US 2019/0153327 A1
(43) Pub. Date: May 23, 2019

(54) WATER-BASED BITUMEN EXTRACTION PROCESSES BASED ON PRIMARY SEPARATION VESSEL FINES LOADING

(71) Applicant: SYNCRUDE CANADA LTD. in trust for the owners of the Syncrude Project as such owners exist now and, Fort McMurray (CA)

(72) Inventors: SHANE HOSKINS, Edmonton (CA); JUN LONG, Edmonton (CA); KEVIN REID, Edmonton (CA); STEPHEN FORWARD, Fort McMurray (CA)

(21) Appl. No.: 15/821,477

(22) Filed: Nov. 22, 2017

(51) Int. Cl. C10G 1/04; G01N 33/24; G01N 15/02

(52) U.S. Cl. CPC C10G 1/047; G01N 15/02; G01N 33/24

(57) ABSTRACT

Fines loading into a primary separation vessel is used to control oil sand ore feed rate and ore fines content to a water-based bitumen extraction plant comprising a slurry preparation unit and to also control the number of primary separation vessels in operation at a bitumen separation plant to minimize operation upsets/excursions and to optimize overall extraction performance.

### FIELD OF THE INVENTION

The present invention relates to an improvement to water-based bitumen extraction processes to avoid process upsets and optimize bitumen recovery by controlling fines loading of primary bitumen separation vessels.

### BACKGROUND OF THE INVENTION

Oil sand ore, as known in the Athabasca region of Alberta, Canada, comprises water-wet, coarse sand grains having flecks of a viscous hydrocarbon, known as bitumen, trapped between the sand grains. The water sheaths surrounding the sand grains contain very fine clay particles. Because of these properties, the bitumen in oil sand can be commercially recovered using a water-based bitumen extraction process.

In the first step of a typical water-based bitumen extraction process, the mined oil sand ore is mixed with heated process water, naturally entrained air and, optionally, caustic (NaOH) or other secondary process aids (SPA) such as sodium citrate, sodium triphosphate, etc., in a slurry preparation unit to form an oil sand slurry. Typical oil sand slurry preparation units include tumblers, cyclofeeders, mix boxes, wet crushing units, and the like. This step is referred to as “oil sand slurry preparation”. The slurry is then conditioned, for example in a tumbler or pipeline, for a prescribed retention time, to initiate a preliminary separation or dispersal of the bitumen and solids and to induce air bubbles to contact and aerate the bitumen. This step is referred to as “slurry conditioning”.

The conditioned slurry is next transported to an extraction plant, where the bitumen is separated from the sand and water. This step is commonly referred to as “bitumen separation”. In particular, the oil sand slurry may be further diluted with flood water and introduced into a large, open-topped, conical-bottomed, cylindrical vessel (termed a primary separation vessel or “PSV”). The diluted slurry is retained in the PSV under quiescent conditions for a prescribed retention period. During this period, aerated bitumen rises and forms a froth layer, which froth overflows the top lip of the vessel and is conveyed away in a launder. Sand grains sink and are concentrated in the conical bottom. They leave the bottom of the vessel as a wet tailings stream containing a small amount of bitumen. Middlings, a watery mixture containing solids and bitumen, extend between the froth and sand layers.

The wet tailings and middlings are separately withdrawn. The wet tailings can be either disposed or combined with the middlings for secondary bitumen recovery in a Tailings Oil Recovery (TOR) vessel. The middlings can also be sent alone to mechanical flotation cells or flotation columns for secondary bitumen recovery. The bitumen recovered from the secondary bitumen recovery process is recycled to the PSV. The froth produced by the PSV is subjected to further froth cleaning, i.e., removal of entrained water and solids, prior to upgrading.

Bitumen recovery is dependent upon a number of factors. One of the primary factors is the quality (or grade) of the oil sand ore. Generally, the lower the grade of ore, which is generally a result of low bitumen content and/or high fines content, the lower the bitumen recovery. Typically, a “low grade” oil sand ore will contain between about 6 to 10 wt. % bitumen with about 25 to 35 wt. % fines. An “average grade” oil sand ore will typically contain at least 10 wt. % bitumen to about 11 wt. % bitumen with less than 25 wt. % fines and a “high grade” oil sand ore will typically contain greater than 11 wt. % bitumen with less than 25 wt. % fines. “Fines” are generally defined as those solids (e.g., silts, clays) having a size less about 44 μm.

To control the feed quality in a water-based extraction process, ore blending (e.g., blending poor quality ores with good quality ores) is commonly used to create a feed with the desirable grade, fines and marine contents. For a given feed quality, a few measures can be used in the conditioning step to optimize bitumen extraction performance, including the control of:
caustic addition based on feed grade and/or fine content;
processing temperatures (this is often limited by utility availability); and
processing density (this is often limited by pumping capacity/line sanding).

These parameters, along with ore quality, govern the bitumen droplet diameter that will enter the PSV. PSV operation relies upon these droplets floating to the froth layer to be recovered. To a first approximation, the rise velocity of the droplets is governed by Stokes Law:

$${U}_{t} = \frac{g ( {ρ}_{p} - {ρ}_{cf} ) {d}_{p}^{2}}{18 {μ}_{cf}}$$

Other than the droplet diameter, the remaining key parameters in Stokes Law are the viscosity and density of the carrier fluid. These can be controlled by adding dilution water to the conditioned oil sand feed prior to it entering the PSV.

For a given bitumen droplet diameter, whether or not it floats to the froth layer largely depends on whether the rise velocity is greater than underflow velocity within the vessel, as shown in the following equation:

U_{b}=U_{uf}+Uₜ

Defining a positive velocity as upwards, a bitumen droplet will only rise to the froth if U_{b} is a positive value. For a given feed density, the vessel underflow velocity is largely governed by the ore feed rate (i.e., the slurry) into the vessel.

In general, all these control measures are based on feed quality information such as grade, fines and marine contents. However, for commercial production of bitumen, due to shovel failures, poor dump sequencing at the crusher and natural variability within the ore body, ore quality can be difficult to control within tight tolerances. In addition, the feed rate to slurry preparation units is often controlled by production requirement (e.g., daily amount of bitumen that needs to be produced) rather than by feed quality. To meet the production requirement, higher feed rates are often needed even if the feed quality is poor. This can lead to upsets or excursions in the overall extraction operation, resulting in very poor performance (low bitumen recovery), or operation shutdown in the worst cases.

For a given feed density, the maximum ore rate that can be fed to a PSV is generally considered to depend on the ore loading of the vessel. The ore loading is defined as follows (Handbook on Theory and Practice of Bitumen Recovery from Athabasca Oil Sands, Vol. II: Industrial Practice, J. Czarnecki et al., ed., 2013):

$$Ore Loading = \frac{Tonnes per Hour of Ore}{Vessel Cross Sectional Area}$$

Correlations of PSV recovery with ore quality and ore loading are common in the art and these correlations are often used to set daily tonnage targets based on the expected ore quality. The ore quality used in these correlations is taken from a database of ore quality that is generated up to years prior to the lease being mined. This database is generated using core-hole samples and these core holes are spaced by approximately 100 m, meaning that the local variation in ore properties is not always available.

More recently, ore grade analyzers and ore fines analyzers have been developed by others and these instruments can be used to monitor the grade and fines content in real time. It should be noted that Syncrude calibrates the performance of these analyzers using the ore quality database measurements mentioned earlier.

It should be clear from the above discussion that ore throughput and ore quality are the keys to successful bitumen extraction. Complex correlations exist between these parameters and vessel recovery, however given the variability in ore processability (even at given grade, fines and marine content), these relationships can carry significant uncertainty and are not ideal to use for online monitoring of performance and for understanding the corrective actions to take when a recovery excursion occurs.

Thus, there is a need in the industry for quantitative rules/guidelines for the control of feed rate to slurry preparation/bitumen extraction plants and to determine the appropriate number of PSVs in operation in order to minimize operation upsets/excursions and to optimize overall extraction performance.

### SUMMARY OF THE INVENTION

It was discovered by the present applicant that water-based bitumen extraction process performance is directly related to the fines loading of the primary separation vessel (PSV). Fines loading is similar to ore loading mentioned earlier, except it takes into account the fines content of the oil sand ore. Fines loading can be determined as follows:

$$Fines Loading = \frac{Tonnes per Hour of Ore \times Fines Content of Ore}{Vessel Cross Sectional Area}$$

Hence, “fines loading” is defined as the amount of fines being processed in a PSV at a given time. Fines loading can be expressed tonnes per hour (TPH) fines per square meter of the vessel cross section area.

Whereas the ore loading only accounts for the throughput in the plant, the fines loading combines the throughout and the ore quality into one parameter. For a given PSV feed density, this parameter provides an indirect indication of both the bitumen rise velocity and the vessel underflow velocity that govern the flotation of bitumen to the froth layer.

The fines content gives an indirect indication of the bitumen rise velocity as it is a proxy for the clay content of the ore. The clay content affects the rise velocity both through increased viscosity and decreased bitumen droplet diameter (due to slime coating). The ore loading gives an indirect indication of the vessel velocity. It is the vessel velocity that can draw poorly floating bitumen to the vessel underflow.

Thus, in one aspect of the present invention, a process for improving a water based bitumen extraction process for an oil sand ore is provided, comprising:
setting an oil sand ore feed rate necessary to produce a desired amount of bitumen;
determining a fines content of the oil sand ore being fed to the water-based extraction process;
determining overall bitumen recovery over a period of time and plotting the recovery against fines loading of at least one primary separation vessel having a cross-sectional area to determine an upper fines loading limitation of the at least one primary separation vessel; and
operating the at least one primary separation vessel below the upper fines loading limitation by adjusting the oil sand ore feed rate to the water-based extraction process.

As used herein, a “water-based bitumen extraction process” comprises three main steps: oil sand slurry preparation, slurry conditioning and bitumen separation in primary separation vessels (PSVs) and is performed at a water-based bitumen extraction plant.

As used herein, “oil sand ore feed rate” means the feed rate of oil sand ore to a water-based bitumen extraction plant. Oil sand ore feed rate can be expressed as tonnes dry oil sand ore per hour (TPH) per vessel vertical cross sectional area.

As used herein “fines content” is defined as the amount of solids having a size less about 44 μm present in an oil sand ore sample, relative to the oil sand sample mass.

It should be noted that fines content is often assumed to mean the fraction of solids having a size less than 44 μm, as compared to the mass of solids only. The fines content is a proxy for the clay content of the ore and this can be approximated through many different parameters that would be obvious to someone skilled in the art (2 micron solids, 5.5 micron solids, MBI, etc.)

In another aspect of the present invention, a process for improving a water-based bitumen extraction process for an oil sand ore is provided, comprising:
setting an oil sand ore feed rate necessary to produce a desired amount of bitumen;
determining a fines content of the oil sand ore being fed to the water-based extraction process;
determining overall bitumen recovery over a period of time and plotting the recovery versus fines loading of a primary separation vessel having a cross-sectional area to determine an upper fines loading limitation of the primary separation vessel; and
determining the number of primary separation vessels having the cross-sectional area necessary to ensure that each primary separation vessel is operating below the upper fines limit.

The “real time” vessel recovery can be monitored by measuring the “real time” bitumen lost to the tailings stream, as measured using the output of a Tailings Oil Analyzer (TOA). The relationship between TOA output and vessel recovery can be obtained by correlating the TOA output to the reconciled mass balance data compiled daily.

In another aspect of the present invention, a method of designing a water-based bitumen extraction plant having at least one primary separation vessel for an oil sand ore mine is provided, comprising:
determining the fines content of oil sand ore present at the mine;
setting a production target for bitumen production per day from the water-based bitumen extraction plant;
setting an oil sand ore feed rate to the water-based bitumen extraction plant necessary to reach the production target; and
sizing the at least one primary separation vessel to provide a desired settling area necessary to avoid fines overloading to the at least one primary separation vessel.

It should be noted that the appropriate fines loading value for an oil sands extraction process will vary depending upon the PSV feed density. The PSV feed density is normally in the range of 1.35-1.45 SG. As defined, Fines Loading only accounts for the dry ore properties and rates and does not account for water addition. At a given fines loading, the density and viscosity of the carrier fluid, as well as the total vessel throughput can be altered by altering the PSV feed density.

### DESCRIPTION OF THE DRAWINGS

FIG. 1 is a schematic showing a typical water-based bitumen extraction process and plant to which the present invention can be applied.

FIG. 2 is a graph showing overall bitumen recovery and primary separation vessel (PSV) fines loading in a typical water-based bitumen extraction process for the period of approximately one (1) year.

FIG. 3 is a graph which plots the variation of carrier fluid viscosity with PSV fines loading (TPH).

FIG. 4 is a graph which plots the vessel velocity with PSV fines loading (TPH).

FIG. 5 is a graph which plots PSV fines loading (TPH) versus number of PSVs on-line.

### DESCRIPTION OF THE PREFERRED EMBODIMENT

The detailed description set forth below in connection with the appended drawings is intended as a description of various embodiments of the present invention and is not intended to represent the only embodiments contemplated by the inventor. The detailed description includes specific details for the purpose of providing a comprehensive understanding of the present invention. However, it will be apparent to those skilled in the art that the present invention may be practiced without these specific details.

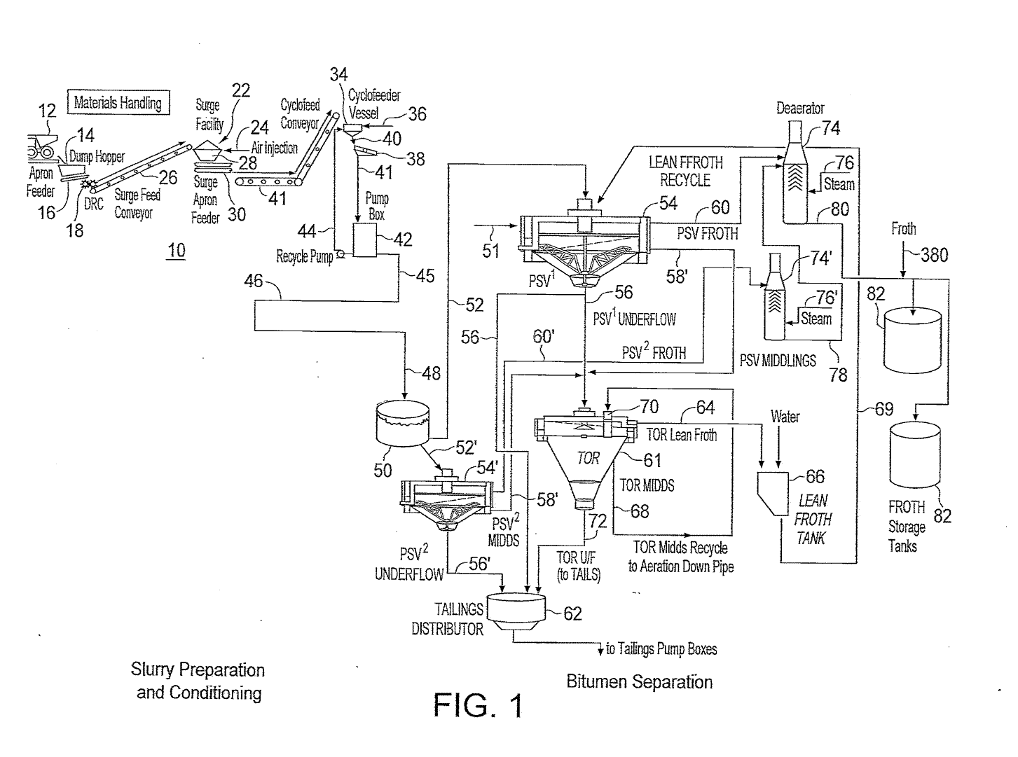

FIG. 1 is a schematic of a typical water-based bitumen extraction plant and process. A water-based bitumen extraction plant generally comprises an oil sand slurry preparation plant, a slurry conditioning apparatus and a bitumen separation plant. In this particular embodiment, oil sand slurry preparation plant 10 comprises mined oil sand being delivered by trucks 12 to a hopper 14 having an apron feeder 16 there below for feeding mined oil sand to a double roll crusher 18 to produce pre-crushed oil sand. Surge feed conveyor 26 delivers pre-crushed oil sand to surge facility 22 comprising surge bin 28 and surge apron feeders 30 there below. Air 24 is injected into surge bin 28 to prevent the oil sand from plugging.

The surge apron feeders 30 feed the pre-crushed oil sand to cyclofeeder conveyer 32, which, in turn, delivers the oil sand to cyclofeeder vessel 34 where the oil sand and water 36 are mixed to form oil sand slurry 40. Oil sand slurry 40 is then screened in screen 38 and screened oil sand slurry 41 is transferred to pump box 42. The cyclofeeder system is described in U.S. Pat. No. 5,039,227. Optionally, oversize lumps from screens 38 are sent to secondary reprocessing (not shown). Oil sand slurry 45 is then conditioned by pumping the slurry through a hydrotransport pipeline 46, from which conditioned oil sand slurry 48 is delivered to slurry distribution vessel 50. A portion of oil sand slurry 44 can be recycled back to cyclofeeder 34.

The bitumen separation plant comprises at least one primary separation vessel, or “PSV”. A PSV is generally a large, conical-bottomed, cylindrical vessel. In the embodiment shown in FIG. 1, slurry is distributed by the slurry distribution vessel 50 (also referred to as “superpots”) to two PSVs 54, 54′ via slurry streams 52, 52′. PSV 54′ is a smaller version of PSV 54, having 0.4 times the volume of the full sized PSV 54. The slurry streams 52, 52′ are commonly diluted with flood water to an appropriate density prior to being fed to the PSVs. Generally, a slurry density of about 1.35 to 1.45 SG is desired. The slurry 52, 52′ is retained in the PSV 54, 54′ under quiescent conditions for a prescribed retention period. During this period, the aerated bitumen rises and forms a froth layer, which overflows the top lip of the vessel and is conveyed away in a launder to produce bitumen froth 60, 60′. The sand grains sink and are concentrated in the conical bottom and leave the bottom of the vessel as a wet tailings stream 56, 56′. Middlings 58, 58′, a mixture containing fine solids and bitumen, extend between the froth and sand layers.

Some or all of tailings stream 56 and middlings 58, 58′ are withdrawn, combined and sent to a secondary flotation process carried out in a deep cone vessel 61 wherein air is sparged into the vessel to assist with flotation of remaining bitumen. This vessel is commonly referred to as a tailings oil recovery vessel, or TOR vessel. The lean bitumen froth 64 recovered from the TOR vessel 61 is stored in a lean froth tank 66 and the lean bitumen froth 64 may be recycled to the PSV feed. The TOR middlings 68 may be recycled to the TOR vessel 61 through at least one aeration down pipe 70. TOR underflow 72 is deposited into tailings distributor 62, together with tailings streams 56, 56′ from PSVs 54 and 54′, respectively. It is understood that a bitumen separation process can be comprised of one or multiple primary separation vessels.

PSV 54 bitumen froth 60 is then deaerated in steam deaerator 74 where steam 76 is added to remove air present in the bitumen froth. Similarly, PSV 54′ bitumen froth 60′ is deaerated in steam deaerator 74′ where steam 76′ is added. Deaerated bitumen froth 78 from steam deaerator 74′ is added to steam deaerator 74 and a final deaerated bitumen froth product 80 is stored in at least one froth storage tank 82 for further treatment. A typical deaerated bitumen froth comprises about 60 wt % bitumen, 30 wt % water and 10 wt % solids.

In this invention, fines loading into the PSV is used to control oil sand ore feed rate and ore fines content to the water-based bitumen extraction plant, e.g., to the slurry preparation unit, and to also control the number of PSVs in operation at the bitumen separation plant to minimize operation upsets/excursions and to optimize overall extraction performance.

### EXAMPLE 1

Determining Fines Loading Limitation

It has been observed that bitumen extraction performance is directly related the fines loading of the primary separation vessel (PSV). Fines loading is defined as the amount of fines being processed in a PSV at a given time. Fines loading is expressed as tonnes of fines per hour per square meter of the vessel's cross section area of the cylindrical top portion of the vessel. The vessel's cross-sectional area is also referred to herein as the “settling area” of the PSV. Fines content in oil sand ore feed can be determined in real time by any means known in the art. For example, K40 measurements can be taken using a K40 analyzer when the oil sand ore is either on conveyor belt 26 or conveyor belt 41, i.e., prior to being fed to the slurry preparation unit 34. It has been shown that there is a proportional relationship between K40 measurements and fines content.

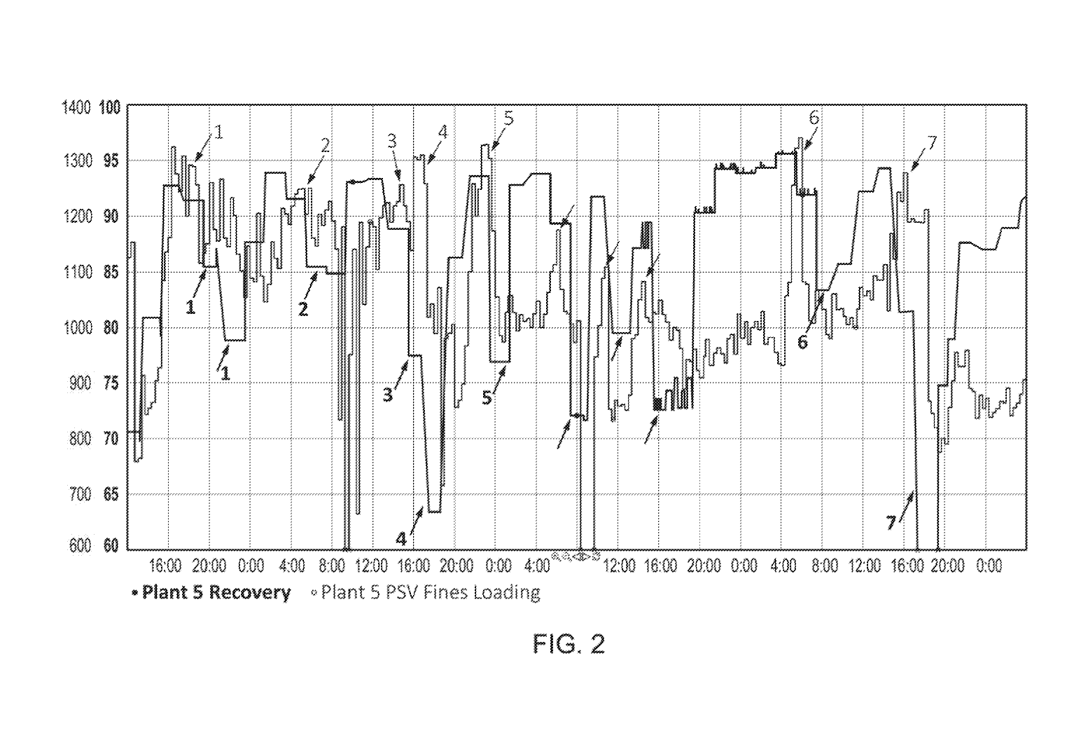

During an extraction operation, overall bitumen recovery was determined at various times during operation and plotted against fines loading at these times. FIG. 2 shows the results of overall bitumen recovery and PSV fines loading in a typical water-based bitumen extraction processes over time. The lines in red and blue represent overall bitumen recovery and fines loading, respectively. It should be noted that there is always a time difference between the feeding of the oil sand ore and the actual bitumen recovery due to the residence times from ore feed to the recovery of the bitumen from the PSVs. As indicated by the blue (upper) arrows and numbers (1 to 7), whenever the fines loading was higher than 1200 TPH (4.25 TPH per m²), there was a corresponding sharp drop later in bitumen recovery (red (lower) arrows and numbers 1 to 7). In general, FIG. 2 shows that a sharp increase in fines loading almost always resulted in a drop in bitumen recovery. In contrast, a decrease in fines loading led to an increase in recovery.

Fundamentally, bitumen flotation and solids settling in a PSV is governed by the well-known Stokes Law:

$${U}_{t} = \frac{g ( {ρ}_{p} - {ρ}_{cf} ) {d}_{p}^{2}}{18 {μ}_{cf}} \\ {U}_{b} = {U}_{uf} + {U}_{t} \qquad ( 1 )$$

This equation shows that the terminal velocity (Uₜ) is governed by the square of the particle/droplet diameter (dₚ), the density difference between the particle/droplet (ρₚ) and the carrier fluid (ρ_{cf}) and the viscosity of the carrier fluid (μ_{cf}). For a given terminal velocity, whether a droplet will float to the froth layer depends on how much fluid is flowing to the underflow of the vessel. If the underflow velocity is greater than the bitumen rise velocity, the bitumen droplet will be drawn out to tailings.

These relationships show that carrier fluid viscosity and density are key parameters in Stokes Law and are of fundamental importance in ensuring optimal recovery in the bitumen flotation process. Although water is used as the slurrying fluid in bitumen extraction, the clay particles within the oil sand ore actually form the carrier fluid with a density and viscosity that differ from that of water alone. The density of the carrier fluid (ρ_{cf}) is a simple function of both the water and clay/fines densities (ρ_{water} and ρ_{fines}) and the fines concentration (γ_{fines}) and is given by:

ρ_{cf}=γ_{fines}ρ_{fines}+(1−γ_{fines})ρ_{water} (2)

A simple, well defined relationship such as equation (2) does not exist for the carrier fluid viscosity. This is due to the fact that, in addition to being a function of the water viscosity and clay concentration, the carrier fluid viscosity is also dependent on the interaction of the clays and this interaction is dependent upon the clay type and water chemistry. A common correlation for carrier fluid viscosity is that given by:

μ_{cf}=exp(12.5C_{f}) (3)

where C_{f} is the volume concentration of fines in the fines-water mixture.

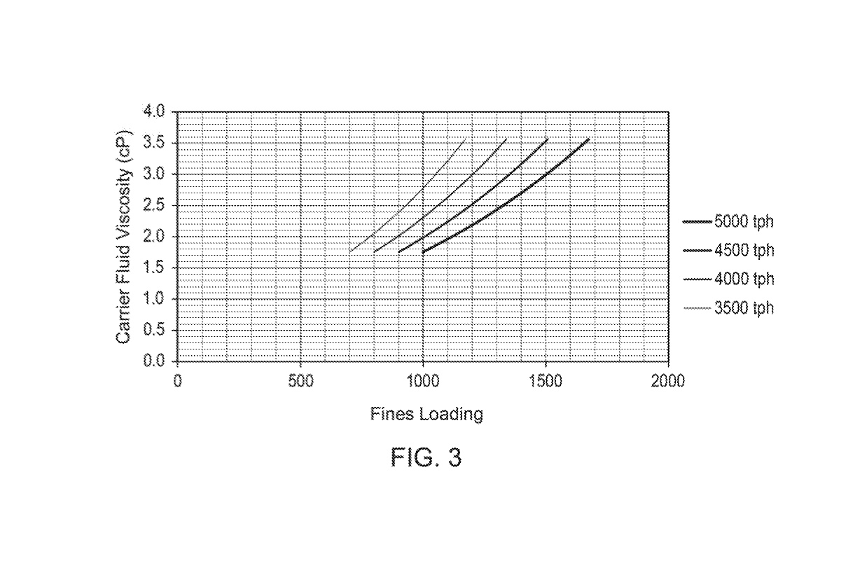

Equations (2) and (3) show that both the carrier fluid density (ρ_{cf}) and viscosity (μ_{cf}) are directly related to and determined by the fines concentrations. These fines concentrations are directly related to the proportion of fines being processed in a PSV, i.e., fines loading. A high fines loading would therefore result in high carrier fluid density and viscosity, thus reducing both the rising velocity of the bitumen droplets and the settling velocity of the solid particles. FIG. 3 shows that when fines loading (TPH) increases, the carrier fluid viscosity (cP) also increases. This leads to poor bitumen-solids separation. As a result, bitumen recovery and over performance are reduced.

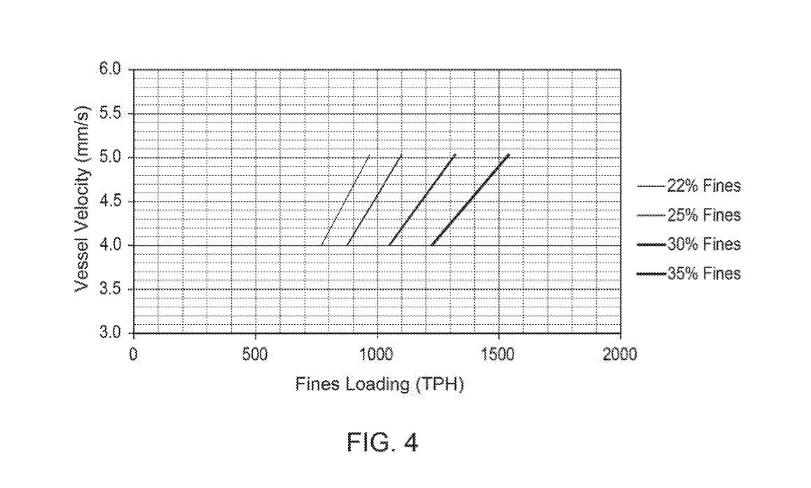

In addition, if fines loading is elevated due to increased ore rate, even at a fixed ore fines content, bitumen recovery can be reduced due to the increased vessel throughput (at a given feed density). The variation of vessel velocity (mm/s) with fines loading (TPH) is shown in FIG. 4.

Hence, a set of PSV fines loading limits can then be determined and used for quantitative control. It was determined that when only caustic was used as the extraction process aid, the fines loading into the PSVs should be lower than 1000 TPH (3.5 TPH per m²) for normal operation and the upper limit is 1200 TPH (4.25 TPH per m²). When a secondary process aid such as sodium citrate or sodium triphosphate is used in combination caustic, the fine loading should be lower than 1100 TPH (4 TPH per m²) for normal operation and the upper limit is 1300 TPH (4.5 TPH per m²).

Thus, by determining fines loading parameters, mine planning can then predict the number of PSVs should be online so as to control the fines loading below the upper limit. Also, efforts should be made to have feed to the extraction plant with fines loading as steady as possible to avoid upsets. Hence, for extraction operation, fines loading should be used to adjust the number of PSVs in operation based on the available feed and its quality so as to control the PSV fines loading under the recommended limits.

Monitoring and Controlling a Water-Based Extraction Process

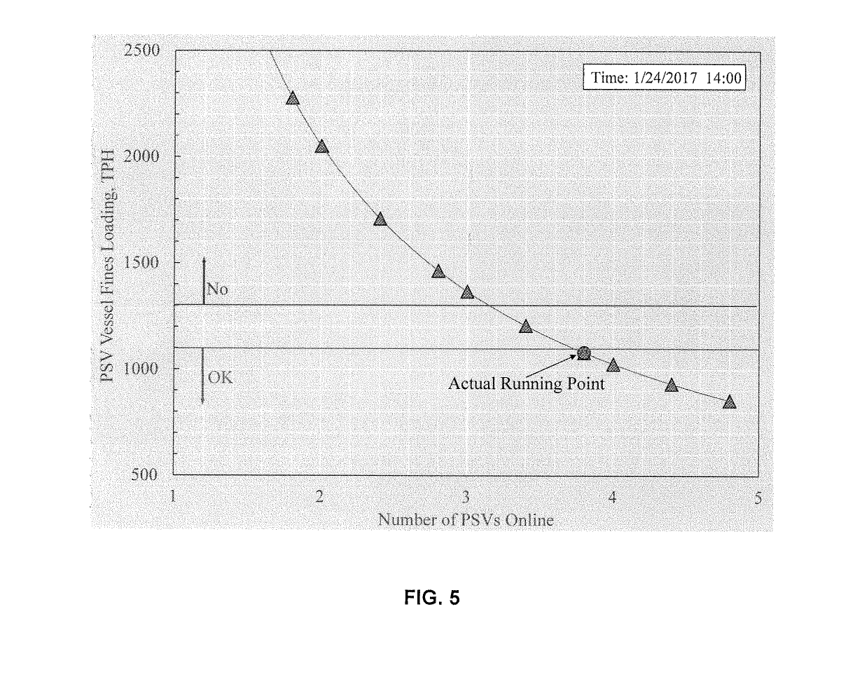

FIG. 5 provides an example which shows how to use fines loading to monitor and control a water-based bitumen extraction process. The lower and upper lines indicate the lower limit and the upper limit, respectively, for fines loading (TPH) into PSV vessels versus the number of PSVs online, i.e., the number of PSVs in operation in the extraction process. When the fines loading is below the green line (≤1100 TPH or 4 TPH per m²), the corresponding number of PSVs will likely be running without problems. When the fines loading is above the green line (>1100 TPH or 4 TPH per m²) but below the red line (≤1300 TPH or 4.5 TPH per m²), the operation of the PSVs should be closely monitored to prevent any potential excursion. When the fines loading is above the red line (>1300 TPH or 4.5 TPH m²), either the feed rate should be reduced or more PSVs should be in operation.

FIG. 5 was developed by live monitoring a water-based bitumen extraction facility operation data. The circle represents a particular point in time which showed that, based on the total oil sand ore feed rate and the average fines content of the feed, the actual fines loading was calculated to be 1080 TPH (3.85 TPH per m²) at that time with 3.8 PSVs (three (3) full sized PSVs and two (2) smaller PSVs, each smaller PSV being equivalent to 0.4 in settling area of a full sized PSV) in operation. It can be seen that the real time fines loading point, shown by the circle, was very close to the green line.

Also shown in FIG. 5 are the calculated fines loadings (the triangles) by assuming different numbers of PSVs online. With the actual oil sand ore feed rate known, which corresponds to the fines loading into the PSV, if there were only three (3) full sized PSVs and one (1) smaller PSV online (i.e., 3.4 PSVs), then the fines loading would be ˜1150 TPH (4.1 TPH per m²) and the PSV operation should be closely monitored. If there were only three (3) full size PSVs online, the fines loading would be above 1200 TPH (4.25 TPH per m²) and this condition should be not be allowed. Thus, the solution would be to either reduce the oil sand ore feed rate/fines content (i.e., fines loading) or have more PSVs online. For example, if the number of PSVs online were increased to four (4) full sized PSVs, the risk of having an excursion could be further reduced.

From the foregoing description, one skilled in the art can easily ascertain the essential characteristics of this invention, and without departing from the spirit and scope thereof, can make various changes and modifications of the invention to adapt it to various usages and conditions.

What is claimed:

1. A process for improving a water-based bitumen extraction process for an oil sand ore, comprising:
setting an oil sand ore feed rate necessary to produce a desired amount of bitumen;
determining a fines content of the oil sand ore being fed to the water-based extraction process;
determining overall bitumen recovery over a period of time and plotting the recovery against fines loading of at least one primary separation vessel having a cross-sectional area to determine an upper fines loading limitation of the at least one primary separation vessel; and
operating the at least one primary separation vessel below the upper fines loading limitation by adjusting the oil sand ore feed rate to the water-based extraction process.

2. The process as claimed in claim 1, wherein
$$Fines Loading = \frac{Tonnes per Hour of Ore \times Fines Content of Ore}{Vessel Cross Sectional Area} .$$

3. A process for improving a water-based bitumen extraction process for an oil sand ore, comprising:
setting an oil sand ore feed rate necessary to produce a desired amount of bitumen;
determining a fines content of the oil sand ore being fed to the water-based extraction process;
determining overall bitumen recovery over a period of time and plotting the recovery against files loading of a primary separation vessel having a cross-sectional area to determine an upper fines loading limitation of the primary separation vessel; and
determining the number of primary separation vessels having the cross-sectional area necessary to ensure that each primary separation vessel is operating below the upper fines limit.

4. The process as claimed in claim 3, wherein
$$Fines Loading = \frac{Tonnes per Hour of Ore \times Fines Content of Ore}{Vessel Cross Sectional Area}$$

5. A method of designing a water-based bitumen extraction plant having at least one primary separation vessel for an oil sand ore mine, comprising:
determining the fines content of oil sand ore present at the mine;
setting a production target for bitumen production per day from the water-based bitumen extraction plant;
setting an oil sand ore feed rate to the water-based bitumen extraction plant necessary to reach the production target; and
sizing the at least one primary separation vessel to provide a desired settling area necessary to avoid fines overloading to the at least one primary separation vessel.
